(19) United States
(12) Patent Application Publication
(10) Pub. No.: US 2008/0227732 A1
(43) Pub. Date: Sep. 18, 2008

(54) Treatment and Control of Severe Infections Including Cystic Fibrosis

(75) Inventor: Manu Chaudhary, Haryana (IN)

Correspondence Address: AMIN HALLIHAN, LLC, 444 NORTH ORLEANS, SUITE 400, CHICAGO, IL, 60610, US

(73) Assignee: VENUS REMEDIES LIMITED, Panchkula, Haryana (IN)

(21) Appl. No.: 11/914,284

(22) PCT Filed: May 8, 2006
(86) PCT No.: PCT/IN06/00158

§ 371 Date: Nov. 13, 2007

(30) Foreign Application Priority Data
May 13, 2005 (IN) 1241/DEL/2005
May 8, 2006 (IN) PCT/IN2006/000158

(51) Int. Cl. A61K 31/7036; A61P 31/00

(52) U.S. Cl. 514/40

(57) ABSTRACT

The present invention provides a pharmaceutical composition comprising antibiotic combination products for delivering two or more different antibiotics simultaneously, wherein the two different antibiotics comprise one which consisting of concentration dependent killing and the other concentration independent killing or time dependant killing activity. The invention has been worked out utilizing pharmacokinetic and pharmacodynamic principles to optimize antibiotic regimen, to improve clinical results and to potentially decrease the development of resistance. Combination of ceftazidime and tobramycin has been evolved to specifically illustrate the invention.

### TECHNICAL FIELD

The invention relates to antibiotic combination products in general. The invention also pertains to parenteral dosage forms of antibiotic combination products and process of producing them for delivering two or more different antibiotics for treatment of diseases in mammals including human being.

### BACKGROUND OF THE INVENTION

Disease like Cystic fibrosis is a hereditary disease that affects a number of organs, particularly the lungs and pancreas. The exocrine glands of a Cystic fibrosis patient secrete abnormally thick mucous, which blocks the patient's bronchi. As a result, many Cystic fibrosis patients have chronic bronchitis; they are also susceptible to pneumonia and other pulmonary infections. In particular, Cystic fibrosis patients are susceptible to Pseudomonas infections.

Unfortunately, the infections of many Cystic fibrosis patients do not respond to the antibiotics traditionally used to treat pulmonary infections. In such a situation, treatments for this disabling disease focuses on alleviating the symptoms of the disease.

Many such similar situations need a practical solution where it becomes clear that the infection is acute, chronic, most probably arising out of resistant bacterial infections, monotherapy is indicated to be ineffective and a better empirical alternative is needed to provide the most probable remedy for obtaining a relief for a patient.

To combat such diseases, the pharmaceutical community has developed a number of different antibiotic agents, which have revolutionized the practice of medicine. Such agents include: amikacin, gentamicin, tobramycin, amoxicillin, amphotericin B, ampicillin, atovaquone, azithromycin, cefazolin, cefepime, cefotaxime, cefotetan, cefpodoxime, cefiazidime, ceftizoxime, ceftriaxone, cefuroxime, cephalexin, chloramphenicol, clotrimazole, ciprofloxacin, clarithromycin, clindamycin, dicloxacillin, doxycycline, erthromycin lactobionate, imipenem, izoniazid, metronidazole, nafcillin, nitrofurantoin, nystatin, penicillin, pentamidine, piperacillin, rifampin, ticarcillin, trimethoprim, vancomycin, and the like. While such agents are effective against most bacteria and therefore useful in the treatment of disease conditions associated with the presence of such bacteria, there is increasing evidence that certain strains of bacteria are becoming resistant to one or more of the known antibiotic agents. Many believe that the emergence of drug resistant bacteria is the result of antibiotic overuse and have thus called for the controlled and limited use of antibiotic agents.

### PRIOR ART

Helm et al (Opthalmology. 1997 May; 104(5):838-43) have reported that combination therapy with intravenous ceftazidime and aminoglycoside may be more effective than single-intravenous agents when used in addition to topical antibiotics and may obviate the need for adjunctive surgical procedures, such as cryotherapy, surgical extirpation, or conjunctival recession.

Kikuchi et al (Jpn J. Antibiot. 1992 July; 45(7):799-808) studied ‘Clinical evaluation of combined therapy of ceftazidime and tobramycin for intractable pulmonary infection mainly caused by Pseudomonas aeruginosa. In an open, multicentre trial, they evaluated utility of the combination therapy and found that the overall efficacy rate in cases where causal organism of pneumonia was P. aeruginosa was 60.0%: but the efficacy rate in moderate cases was 100% and that in severe cases was 45.5%. In cases where causal organism was gram negative bacilli, the overall efficacy rate was 72.2% with 100% efficacy rate among moderate cases and 68.8% among severe cases. In the cases with chronic respiratory tract infections caused by P. aeruginosa, the efficacy rate was 82.6% and the eradication rate was 65.2%.

Above results very clearly show that combination therapy of ceftazidime and tobramycin is useful for intractable pulmonary infections caused by P. aeruginosa. Efficiency of curing 45.5% or more of severe cases and 72.2 to 100% of moderate cases in above clinical trials clearly indicate existence of synergistic action between these two antibiotics.

Jacobs et al (Infection. 1993 July-August; 21(4):223-8) studied the efficacy and safety of ceftazidime versus ceftazidime plus tobramycin in the treatment of febrile children (range 8 months to 18 years) with neutropenia secondary to cancer chemotherapeutic agents. Of the evaluable 89 patients, 45 received ceftazidime and 44 received ceftazidime plus tobramycin for 5 to 10 days. At the end of therapy, 30 (67%) of the 45 ceftazidime-treated patients were clinically cured compared with 38 (86%) of 44 combination-treated patients. The results show that in difficult cases of febrile neuropenic children, combination therapy of ceftazidime plus tobramycin is a better alternative to monotherapy of ceftazidime.

Double beta-lactam regimen compared to an aminoglycoside/beta-lactam regimen as empiric antibiotic therapy for febrile granulocytopenic cancer patients was studied by Joshi et al (Support Care Cancer. 1993 July; 1(4):186-94). Both regimens produced excellent serum bactericidal levels (C+/−T geometric mean peak 1:170; C+P peak 1:137) against gram-negative but not gram-positive pathogens (1:4; 1:7 respectively) that had caused bacteremia. Emergence of resistance and significant coagulopathy and/or bleeding did not occur during therapy. The incidence of secondary infections in patients with profound (<100/microliters) sustained granulocytopenia was lower in the C+/−T group (P=0.04).

A randomized study of ceftazidime compared to ceftazidime and tobramycin for the treatment of infections in cancer patients was done by Fainstein V et al (J Antimicrob Chemother. 1983 July; 12 Suppl A: 101-10). They highlighted that ceftazidime should be combined with an agent active against Gram-positive pathogens in neutropenic patients. The overall response rate in 83 episodes of infection treated with ceftazidime alone was 60% and 73% in those who received the combination. The overall response rate in septicaemia was 75% with ceftazidime alone and 85% with the combination. Pneumonias in neutropenic patients responded equally well. However, patients with adequate neutrophil counts responded better to the combination than to single-agent therapy. The rates of superinfection and toxicity were very low.

Balke et al (Eur J Clin Microbiol Infect Dis. 2006 January; 25(1):25-30) reported that the determination of synergistic effects of antimicrobial drug combinations can lead to improved therapeutic options in the antibiotic treatment of Cystic fibrosis patients who are chronically infected with multiresistant Pseudomonas aeruginosa isolates. The rate of synergy was higher for the antibiotic combination of ceftazidime and tobramycin (28.8% of the Cystic fibrosis strains) than for the combination of meropenem and tobramycin.

Cantón et al (Clinical Microbiology & Infection, Volume 11, Number 9, September 2005, pp. 690-703(14) studied antimicrobial therapy for pulmonary pathogenic colonization and infection by Pseudomonas aeruginosa in Cystic fibrosis patients and found patients with acute infection should be treated for 14-21 days with high doses of two intravenous antimicrobial agents, with or without an inhaled treatment during or at the end of the intravenous treatment. Maintenance treatment after development of chronic P. aeruginosa infection/colonization (pathogenic colonization) in stable patients (aged >6 years) should be with inhaled tobramycin (300 mg twice-daily) in 28-day cycles (on-off) or, as an alternative, colistin (1-3 million units twice-daily. Moderate and serious exacerbations can be treated with intravenous ceftazidime (50-70 mg/kg three-times-daily) or cefepime (50 mg/kg three-times-daily) plus tobramycin (5-10 mg/kg every 24 h) or amikacin (20-30 mg/kg every 24 h) for 2-3 weeks.

Hollander et al (Antimicrob Agents Chemother. 1997 January; 41(1):95-100.) in “Synergism between tobramycin and ceftazidime against a resistant Pseudomonas aeruginosa strain, tested in an in-vitro pharmacokinetic model.” reported that there is synergism between tobramycin and ceftazidime at declining antibiotic concentrations below the MIC, resulting in a pronounced killing of a resistant Pseudomonas strain. Infections due to resistant Pseudomonas strains could possibly be treated by a synergistic combination of these drugs.

Chen & Zabransky (Diagn Microbiol Infect Dis. 1987 February; 6(2):157-64.) in their study reported. synergistic or additive effects in the tobramycin-ceftazidime combination against tobramycin-resistant strains of P. aeruginosa and P. maltophilia, and with all tobramycin combinations against tobramycin-susceptible strains of P. aeruginosa using the checkerboard technique.

Zelenitsky et al (Diagn Microbiol Infect Dis. 2004 May; 49(1):67-70.) in their studies showed that antibiotic sequence had a significant and class dependent effect on antibacterial response.

Hollander et al. (Antimicrob Agents Chemother. 1998 April; 42(4):744-8.) in their studies concluded that for combination therapy with tobramycin and ceftazidime the T>FICi is the parameter best predictive of efficacy and that the E-test for susceptibility testing of combination therapy gives promising results. These new pharmacodynamic parameters for combination therapy promise to provide better insight into the rationale behind combination therapy.

Above mentioned prior art methods of using two antibiotics in in-vivo studies in combination with the in-vitro studies show the promise of use of combination antibiotics for treatment of drug resistant infections. However, administering more than one antibiotic for a combination treatment has several limitations, disadvantages as well as defects. One feature of the references stated above is that each drug of the combination used therein was individually administered one after the other without specific or predetermined ratio. Such administration and also the co-administration as mentioned in case of some of the above references have a number of disadvantages. The individual administration of the ceftazidime and tobramycin components of drugs described in the prior art failed to solve the treatment problem satisfactorily because of following reasons:
1. Drugs mentioned as the combinations used in the multiple drug treatment were administered one after the other individually in doses which were not optimal different doses than the invention.
2. These drugs were not available in a premixed compositions as one drug.
3. A further complexity is involved in administration of the drug as more number of pricks is required and the time of administration is also long.
4. Treatment time is prolonged to about 14-21 days in case of individual administration of these drugs.
5. Cost to the patient is higher due to increased hospitalization time.
6. The failure rate is higher due to inconsistency of dose. The components are administered either in equal proportions or the ratio is undefined and not fixed. e.g Cantón et al used inhaled tobramycin (300 mg twice-daily), Blumer et al, in Chest, 2005; used ceftazidime (5 mg/kg to 2 g q8h), which was administered with IV tobramycin (at a serum peak of > or =8 microg/mL and a trough of <2 microg/mL); meaning thereby that there were no fixed doses available for treatment of such kind of infections.
7. Use of other route like use of tobramycin as inhaler with parenteral route is adopted in some cases.
8. Due to non availability of pharmaceutically effective fixed dose composition at fixed intervals, chances of development of resistance are very high in case of prior art methods described. In the absence of a predetermined dosage schedule of known efficacy, there is scope for arbitrary choice of dosage leading to a treatment variation from case to case, which could most probably be sub-optimal only.
9. Co-administration has to be done very carefully as two individual components are not chemically compatible with each other and there are several precautions that have to be followed in case of prior art such as use of different syringes for individual component, control on time of administration of two drugs and the like.

These limitations, disadvantages and defects are removed/circumvented in this invention.

Advantages of combination therapy in present invention include a wider range of modes of action, improved efficacy of the composition on account of additive effect, synergy and reduction of resistant organisms/rate of super-infection.

It is generally accepted that a dead bug cannot mutate and pass on resistance. The two measures of this are Maximal Bacterial Concentration (MBC) as required to kill the Bacterium and MPC which is the Mutant Prevention Concentration (cf. Tulkins, Mouton ISAP Conference at ECCMID, April 2001). The MPC may be seen as an antibiotic concentration that will quickly kill all bacteria and kill bacteria with decreased susceptibility. The parameters of the drug include, without limitation, pharmacokinetic and pharmacodynamic parameters and the derived MBC or MPC concentrations. The MBC or MPC concentrations are either calculated or measured.

The invention is based at least in part on the realization that pharmacokinetic data for a particular antibiotic drug can be used to derive infusion characteristics for that drug which can be programmed into a delivery system for that particular drug. We anticipate that use of the system will mean less antibiotic is required per therapeutic treatment and that treatment times will be shorter.

It is an embodiment of present invention wherein different antibiotics selected on the basis of their proven better combined efficacy in published clinical trials are made more efficacious and more convenient to administer by inventing the most efficacious combination based on in-vitro experimentation and by inventing fixed dose combinations with defined dosage schedule, which are compatible with each others in an injectable dosage form which can be given intramuscularly or intravenously as a parenteral route treatment. In this fashion, many combinations of antibiotics are possible and all of them are included in this invention. An illustration of the invention is provided by a fixed dose combination of ceftizidime and tobramycin, a combination which has already been demonstrated by clinical trials reported so far to be synergistically effective against a very wide range of pathogens.

Treatment instituted before knowing the aetiology and antimicrobial sensitivities is empirical. Therefore, present invention provides the desired empirical therapy for control of widest known range of all bacterial infections. Such combinations of the invention have shown enhanced efficacy of the combination even in in vitro sensitivity test and clinical trials are in progress.

For example, enterococci that are resistant to a vast array of antimicrobial drugs, including cell wall active agents, aminoglycosides, penicillin, ampicillin, and vancomycin, have been observed in in vitro tests to be better controlled by the inventive synergistic combination of tobramycin combined with ceftazidime at critical concentrations of this invention

The approach of this invention as applied to ceftidizime and tobramycin combination can potentially be useful for similar combinations of two or more antibiotics shown by published clinical trials to be synergistically useful when administered as separate doses. However, to make administration of multiple antibacterial agents possible as one injectable pharmaceutical composition and also as a method of treatment and prevention for infective conditions, it is necessary to ensure that:

they are safe and chemically compatible to each other
they can be administered easily without posing any medical hazard,
they provide effective treatment of the hospitalized patient for the treatment of bacterial infections to optimize antibiotic regimen, to improve clinical condition and to potentially decrease the development of resistance.
they provide efficacy against a wide variety of infectious organisms,
they have a potential to administer a lower dose of a therapeutic agent while still providing efficacy,
they have a potential to administer a higher dose of an antibacterial agent without increased side effects.
they ensure improvement of the therapeutic index of an active agent while decreasing its general toxicity and minimizing the risk of systemic effects
They decrease the chances of super infection.
Meeting above requirements is not possible by a simple approach of mixing of ingredients, but it is necessary to invent a composition of the target antibacterial agents which shall satisfy all above criteria.

The inventiveness about this patent lies in the following:
1. The two drugs have been combined as one drug for the first time as dry powder for injection and liquid solution for injection as a fixed dose combination.
2. Although, in general, the cephalosporins and aminoglycosides are non-compatible with each other, it is a finding of this invention that they are compatible in presence of only a specific concentration of stabilizing agents and other components.
3. The dose deciding was the most innovative step involved in it as Tobramycin if given in higher doses can be nephrotoxic and prove fatal. The dose which was found out to be safe was 60 mg Tobramycin with 500 mg Ceftazidime; 120 mg Tobramycin with 1.0 g Ceftazidime and 180 mg Tobramycin with 2.0 g Ceftazidime.
4. The combination proves synergistic and is more effective than either of the drug alone.
5. Both the ingredients selected have Pharmacokinetic and Pharmacodynamic compatibility in ratios identified in the invention and specified dosage schedules.
6. The treatment time is reduced and cost to patient is very less.
7. Treatment time is reduced by 25% to 30% as compared to prior art
   Accordingly, the objects of the present invention are described as below:
   Accordingly an object of the present invention is to provide pharmaceutical compositions that are safe, that have efficacy against a wide variety of infectious organisms, and to provide a composition that is useful in providing effective treatment against non-ocular infective conditions of a multi drug resistant bacterium.

Yet another object of the present invention is to provide a method of treatment of non-ocular infective conditions that ensures rapid therapeutic delivery of therapeutic agent(s) to the site of the infective condition.

Further object of the present invention is to provide pharmaceutically effective dose for parenteral administration for hospitalized patients with acute or serious non-ocular infections.

Still another object of the present invention is to provide dosage schedules that have a potential to provide effective treatment without increased side effects like nephrotoxicity.

A further objective of the present invention is to provide a process of making pharmaceutical compositions of the present invention.

A still further objective of the present invention is to provide a chemically compatible stable formulation, which is easy to administer.

A still another objective of the present invention is to provide less treatment period for curing in the patients

A still further objective of the present invention is to provide cost effective treatment with decreased hospitalization period.

A still another objective of the present invention is to provide timely and adequate treatment for critically ill ICU patients where doctor cannot wait for culture reports to come.

A still further objective of the present invention is to administer a higher dose to chronically ill patients with least probability of increased side effects

A still further objective of the present invention is to administer a lower dose of combination with better efficacy than either of the two individually administered drug against specified bacterium.

A still another objective of the present invention is to ensure improvement of the therapeutic index of an active agent while decreasing its general toxicity and minimizing the risk of systemic effects.

A still another objective of the present invention is to ensure a Fixed Dose Combination product with better pharmacokinetic and Pharmacodynamic compatibility.

In the following are given a brief summary of the invention, details of the invention and examples which illustrate working of the invention. It is to be understood that the invention is not limited to the particular embodiments of the invention described below, which are for limited purpose of illustrating operation of this invention, as variations of the particular embodiments obvious to a person skilled in the art may be made and still fall within the scope of the appended claims. It is also to be understood that the terminology employed is for the purpose of describing particular embodiments, and is not intended to be limiting. Instead, the scope of the present invention will be established by the appended claims. Further, in this specification and the appended claims, the singular forms “a,” “an,” and “the” include reference to their plural forms too unless the context clearly dictates otherwise. Thus, for example, “a beta-lactam antibiotic” also includes one or more of all beta-lactam antibiotics; “a stabilizer” includes all the known stabilizers and includes use of only one or more than one stabilizers in the same composition; a mention of “a disease” includes mention of one or more diseases and the like. Further, unless defined otherwise, all technical and scientific terms used herein have the same meaning as commonly understood to one of ordinary skill in the art to which this invention belongs.

### BRIEF SUMMARY OF THE INVENTION

This invention discloses a process of producing a pharmaceutical composition and the ingredients of the composition itself that is suitable for parenteral injection for use as antimicrobial in a human being, comprising a dry powder/liquid dosage form, of a synergistic or a more effective combination of antibiotics, at least one of which acts in a concentration-dependent manner, comprising preferably an aminoglycoside antibiotic or a pharmaceutically acceptable salt thereof, which is compatible with at least another antibiotic which acts in a time-dependent manner, comprising preferably a beta-lactam antibiotic or a pharmaceutically acceptable salt thereof added in a form and in a concentration which shall reach after injection cmax in serum almost simultaneously with a plasma half life of about 2 hours; with or without addition of one or more of a stabilizing agent, a soothing agent, a buffering agent, an adjuvant, an antiseptic agent, a chelating agent, an anesthetic agent and/or an additive contributing an improvement in performance of the composition.

One such combination of ceftidizime and tobramycin has been investigated and standardized in details that comprises of tobramycin or pharmaceutically acceptable salt thereof, 20 to 220 mg as free acid, and ceftazidime or pharmaceutically acceptable salt thereof, 250 mg to 2 gram as free acid, taken in weight/weight proportion of tobramycin: ceftazidime in the range of 1:8.33 to 1:11.2. The composition is sealed under sterile conditions in a sealed container, preferably having a small headspace filled with nitrogen. Intramuscular or intravenous infusion of the composition of the invention provides a method of treating several disease conditions involving an acute and resistant bacterial infection arising out of several diseases including but not limited to comprise of acute pulmonary exacerbations (APEs), febrile neutropenia, Cystic fibrosis, other pulmonary bacterial infections, Lower Respiratory Tract Infections including pneumonia.

### DETAILED DESCRIPTION OF INVENTION

This invention relates to an antibiotic composition utilizing pharmacokinetic and pharmacodynamic principles and the uses thereof. The composition delivers two antibiotics one of which is a concentration dependent killing antibiotic and the other is a concentration independent killing antibiotic or time dependent killing antibiotic. More particularly, this invention relates to a composition for the parenteral delivery of two different antibiotics, their dosage schedule and the uses thereof.

The terminology “concentration dependent killing antibiotic” means an agent that shows concentration dependent bactericidal activity in vitro; the higher the antibiotic concentration the greater is the extent of activity.

The terminology “concentration independent killing antibiotic” means antibiotics whose bactericidal activity is dependant on time for which it is available at the site of injection for action against the bacterium and not on concentration.

In many cases, it is desirable to employ two different antibiotics in the treatment of a bacterial infection, in that such antibiotics may have complementary mechanisms of action that facilitate broad-spectrum coverage, bactericidal activity and potential synergistic effects, and to minimize the development of resistance during treatment of the severe or acute bacterial infections.

As non-limiting representative examples of “concentration independent killing antibiotic beta-lactam that can be used in working of this invention include, without being limited to, following antibiotics or their pharmaceutically acceptable and effective salts thereof of benzylpenicillin, phenoxymethylpenicillin, phenethicillin, propicllin, ampicillin, methicillin, oxacillin, cloxacillin, flucloxacillin, dicloxacillin, hetacillin, talampicillin, bacampicillin, lenampicillin, amoxicillin, ciclacillin, carbenicillin, sulbenicillin, ticarcillin, carindacillin, carfecillin, piperacillin, mezlocillin, aspoxicillin, cephaloridine, cefazolin, cefapirin, cephacetrile, ceftezole, cephaloglycin, cephalexin, cephalexin, cefatrizine, cefaclor, cefroxadine, cefadroxil, cefamandole, cefotiam, cephalothin, cephradine, cefuroxime, cefoxitin, cefotaxime, ceftizoxime, cefinenoxime, cefodizime, ceftriaxone, cefuzonam, ceftazidime, cefepim, cefpirome, cefozopran, cefoselis, ceflurenam, cefoperazone, cefpimizole, cefpiramide, cefixime, cefteram pivoxil, cefpodoxime proxetil, ceftibuten, cefetamet pivoxil, cefdinir, cefditoren pivoxil, cefcapene pivoxil, cefsulodin, cefoxitin, cefinetazole, latamoxef, cefotetan, cefbuperazone, cefminox, flomoxef, aztreonam, ertapenem, carumonam, imipenem, panipenem, meropenem, viapenem, faropenem, ritipenem acoxil, or mixtures thereof, which are non-protein synthesis inhibiting.

As non-limiting representative examples of “concentration dependent killing antibiotic aminoglycoside or a pharmaceutically accepted salt thereof which can be used in working of this invention include one or more of, gentamicin, amikacin, tobramycin; erythromycin, streptomycin, lincomycin; tetracycline, doxycycline, chlortetracycline, minocycline; linezolid; fusidic acid; kanamycin, netilmicin and chloramphenicol and other protein synthesis inhibiting antibiotics and a pharmaceutically acceptable salt thereof which are protein synthesis inhibiting.

In a preferred embodiment such two antibiotics are delivered simultaneously.

The present invention is directed in particular to a new and improved product that delivers tobramycin or a pharmaceutically acceptable salt thereof, and ceftazidime or a pharmaceutically acceptable salt thereof, in a specific dose for the treatment of bacterial infections caused by susceptible bacteria for the treatment of severe and acute infections.

In formulating the antibiotic composition of the present invention, which contains different strengths of parenteral dosage form in liquid/dry powder for reconstitution before injection, as hereinabove described, the first antibiotic, a concentration dependent killing antibiotic, such as tobramycin, generally forms about 9-12 percent of a concentration independent killing antibiotic such as ceftazidime by weight.

In formulating the antibiotic composition of the present invention, which contains different strengths of parenteral dosage form in liquid/dry powder for reconstitution before injection, as hereinabove described, more suitably, the second antibiotic i.e. a concentration independent killing antibiotic or time dependent killing antibiotic such as ceftazidime generally forms about 800 percent to about 1200 percent of a concentration dependent killing antibiotic such as tobramycin by weight.

In formulating the antibiotic composition of the present invention, which contains different strengths of parenteral dosage form in liquid/dry powder for reconstitution before injection, as hereinabove described, most suitably, the second antibiotic, a concentration independent killing antibiotic or time dependent killing antibiotic such as ceftazidime generally forms about 89-91 percent of the combination product by weight; whereas a concentration dependent killing antibiotic such as tobramycin generally form about 11-9 percent of the combination product by weight.

The antibiotics may be in the form of a pharmaceutically acceptable salt. Pharmaceutically acceptable salts refer to salts which can be generally used as salts of an antibiotic in pharmaceutical industry, including for example, salts of sodium, potassium, calcium and the like, and amine salts of procaine, dibenzylamine, ethylenediamine, ethanolamine, methylglucamine, taurine, and the like, as well as acid addition salts such as hydrochlorides, sulphates and basic amino acids and the like.

The invention is embodied into the antibiotic composition of this invention in one or more of the following aspects:
1. in determining the fixed proportions of tobramycin and ceftazidime in the composition so as to minimize the toxic effects of high doses of individual components
2. in the use of one/more stabilizing/other agents in general and the use of L-arginine and/or sodium carbonate in particular,

In one embodiment of the invention, the combination of tobramycin to ceftazidime to arginine and or sodium carbonate is in ratio of 1:7:1.

In other embodiment, the combination of tobramycin to ceftazidime to arginine is in ratio of 1:8:1 to 1:10:1.

In yet another embodiment, the combination of tobramycin to ceftazidime to arginine is in ratio of 1:9.8:1.3.

This invention also includes a process of making a sterile blended liquid/dry powder composition. In one embodiment the invention provides a process for the manufacture of a pharmaceutical composition that can be reconstituted by addition of a compatible reconstitution diluent prior to parenteral administration and, if desired, diluted with a compatible diluent prior to parenteral administration; which comprises effective amounts of (a) tobramycin or a pharmaceutically acceptable salt thereof preferably sulphate salt, (b) ceftazidime or a pharmaceutically acceptable salt thereof preferably pentahydrate salt and a stabilizing agent in the form of l-arginine and/or sodium carbonate. In this case, the appropriate solvent is usually added to sterile blended composition, which preferably is distilled water for injection, but is not limited thereto in accordance with the invention.

In another embodiment for a liquid dosage form, both the active ingredients are dissolved in an appropriate medium and the resulting solution is sterilized and filtered followed by filling in an appropriate ampoule or vial, and sealing. Liquid injection may contain additives such as soothing agents which have local anesthetic effect, such as procaine hydrochloride, xylocaine hydrochloride, benzyl alcohol and phenol, antiseptic agents such as benzyl alcohol, phenol, methyl or propyl paraben and chlorobutanol, buffering agents such as a sodium salt of citric acid, phosphoric acid, acetic acid, solution adjuvants such as arginine hydrochloride, Sodium Meta Bi Sulphite, stabilizing agents such as L-cysteine, L-methionine, L-histidine, and chelating agents, if required.

In a preferred embodiment parenteral dosage form of both the antibiotics have almost the same kinetics.

In another aspect, the present invention is directed to treating a bacterial infection by administering to a host preferably mammal more preferably human beings in need thereof, an antibiotic product as herein above and hereinafter described.

In another aspect, the present invention is directed to treating a bacterial infection caused by Aerobes, Gram negative: Citrobacter spp., including Citrobacter freundii and Citrobacter diversus; Enterobacter spp., including Enterobacter cloacae and Enterobacter aerogenes; Escherichia coli; Haemophilus influenzae, including ampicillin-resistant strains; Klebsiella spp. (including Klebsiella pneumoniae); Neisseria meningitidisi; Proteus mirabilis; Proteus vulgaris; Pseudomonas spp. (including Pseudomonas aeruginosa); and Serratia spp. Aerobes, Gram positive: Staphylococcus aureus, including penicillinase- and non-penicillinase-producing strains; Streptococcus agalactiae (group B streptococci); Streptococcus pneumoniae; and Streptococcus pyogenes (group A beta-hemolytic streptococci). Anaerobes: Bacteroides spp. Acinetobacter spp., Clostridium spp., (not including Clostridium difficile), Haemophilus parainfluenzae, Morganella morganii (formerly Proteus morganii), Neisseria gonorrhoeae, Peptococcus spp., Peptostreptococcus spp., Providencia spp. (including Providencia rettgeri, formerly Proteus rettgeri), Salmonella spp., Shigella spp., Staphylococcus epidermidis, and Yersinia enterocolitica., methicillin-resistant staphylococci, Streptococcus faecalis and many other enterococci, Listeria monocytogenes, Campylobacter spp., or Clostridium difficile.

Thus, in accordance with an aspect of the present invention, there is provided a fixed dose antibiotic combination product that has contained therein as parenteral dosage form, which initiates release of antibiotic at same time and wherein includes at least a concentration dependent killing antibiotic such as tobramycin sulphate, a concentration independent killing antibiotic or time dependent killing antibiotic such as ceftazidime pentahydrate along with 1-arginine and/or sodium carbonate.

In another aspect, the present invention relates to a product that delivers tobramycin or a pharmaceutically acceptable salt thereof, along with ceftazidime or a pharmaceutically acceptable salt thereof, in a specific dose for the treatment of bacterial infections like Cystic Fibrosis, Lower Respiratory Tract Infections, including pneumonia, Skin and Skin Structure Infections, Urinary Tract Infections, both complicated and uncomplicated, Bacterial Septicemia, Bone and Joint Infections, Gynecologic Infections, including endometritis, pelvic cellulitis, and other infections of the female genital tract, Intra abdominal Infections, including peritonitis and polymicrobial infections, Central Nervous System Infections, including meningitis etc.

In accordance with a preferred embodiment elimination of tobramycin and ceftazidime is principally via renal excretion with an average (±SD) half-life of 2.0 (±0.3) hours and the mean renal clearance of approximately 100.0 (±10.0) mL/min and calculated plasma clearance is approx-115.0 ml/min in healthy volunteers.

In accordance with one preferred embodiment of the invention, the average period of the treatment with tobramycin and ceftazidime equaled to 7 days (5 to 10).

In general, the invention is available as sterile blend of two or more dry powders in fixed ratios to be reconstituted before injection with suitable solvent. However, it can also be formulated and sealed as liquid composition.

In a preferred embodiment the administration of the antibiotic product is a concentrate that is diluted before administration in suitable infusions; such as Sterile Water for Injection, 0.9% Sodium Chloride, 5% Dextrose Injection.

In an embodiment of this invention, the composition of this invention is sterile packed in a sealed container which has an interior comprising a fill volume occupied by the suitable solvent and a headspace volume occupied by a micro atmosphere having a nitrogen pressure of not more than about 5%, wherein the ratio of reconstituted fill volume to headspace volume is not less than about 1:1.

In another embodiment of this invention, wherein a pharmaceutically effective unit/multiple dose of said combination is provided in a sealed airtight container which is selected from the group consisting of a vial, a mono vial, an ampoule, a syringe, a packet, a pouch and an auto-injector, wherein said container has a head space volume sufficient for introduction of appropriate volume of an aqueous solvent sufficient to form a unit/multiple dose in the form of an appropriate reconstituted solution of said combination.

In yet another embodiment of this invention, wherein said pharmaceutical composition is packed in a sealed container wherein said container has a headspace sufficient for introduction of a volume of aqueous solvent sufficient to form a concentrated solution of said pharmaceutical composition.

The antibiotic composition of the present invention may be administered by the following routes of administration: parenteral, by intramuscular or intravenous administration and the preferred regimen is that the product is administered 2-3 times for intramuscular injection and intravenous infusion over a 24 hour period.

Examples which illustrate various embodiments of this invention are given in the following, without limiting the scope of invention as claimed.

### EXAMPLE 1

### Bacterial Susceptibility Test

This test was performed by disc diffusion test for ceftazidime and tobramycin on Müeller-Hinton Agar medium purchased from Hi Media. The medium was prepared and used as per manufacturer's instructions. Ceftazidime alone, Tobramycin alone and the combination of Ceftazidime and Tobramycin, on different micro-organisms was taken. Different concentrations of the antibiotics or their combinations were selected mentioned to as highest (10 mg/ml Ceftazidime, 1.2 mg/ml Tobramycin and 10 mg/ml+1.2 mg/ml as combination of the two), high (1 mg/ml Ceftazidime, 0.12 mg/ml Tobramycin and 1 mg/ml+0.12 mg/ml as combination of the two), low (0.1 mg/ml Ceftazidime, 0.012 mg/ml Tobramycin and 0.1 mg/ml+0.012 mg/ml as combination of the two) and lowest (0.01 mg/ml Ceftazidime, 0.001 mg/ml Tobramycin and 0.01 mg/ml+0.001 mg/ml as combination of the two in the data and discs. Zone size was determined in mm. The activity of ceftazidime and tobramycin is best seen in P. auerignosa, E. coli, Klebsiella pneumoniae, Staphylococcus (MSSA), C. albicanus, MRSA. The three strength tested were 560 mg (500 mg ceftazidime and 60 mg tobramycin), 1120 mg (1000 mg ceftazidime and 120 mg tobramycin) and 2180 mg (2000 mg ceftazidime and tobramycin 180 mg). For each concentration the three zones were observed i.e for combination, Ceftazidime alone and Tobramycin alone and tested for efficacy against various types of micro-organisms.

### Results are given in following Table-1

TABLE 1
Bacterial susceptibility data on Ceftazidime and Tobramycin

| | | Highest | | | High | | | Low | | | Lowest | | |
|---|---|---|---|---|---|---|---|---|---|---|---|---|---|
| S. NO | ORGANISM | (mg/ml) | | | (mg/ml) | | | (mg/ml) | | | (mg/ml) | | |
| | | Concentration 1.12 g | | | | | | | | | | | |
| | | C | T | I | C | T | I | C | T | I | C | T | I |
| | | 10.0 | 1.2 | 11.2 | 1.0 | 0.12 | 1.12 | 0.1 | 0.01 | 0.112 | 0.01 | 0.001 | 0.011 |
| 1 | MRSA | 22.39 | 33.22 | 33.42 | 12.53 | 22.45 | 26.48 | 0 | 10.24 | 15.54 | 0 | 9.89 | 13.68 |
| 2 | E. coli | 47.34 | 30.30 | 50.26 | 42.10 | 25.02 | 44.46 | 35.49 | 22.16 | 41.06 | 33.34 | 14.35 | 34.30 |
| 3 | P. aurignosa | 48.34 | 30.31 | 56.26 | 44.10 | 23.02 | 49.46 | 32.48 | 19.16 | 40.06 | 29.34 | 11.35 | 34.82 |
| | | Concentration 560 mg | | | | | | | | | | | |
| | | C | T | I | C | T | I | C | T | I | C | T | I |
| | | 5.0 | 0.6 | 5.6 | 0.5 | 0.06 | 0.56 | 0.05 | 0.006 | 0.056 | 0.005 | 0.0006 | 0.0056 |
| 1 | MRSA | 21.19 | 32.39 | 33.29 | 12.88 | 23.89 | 24.89 | 0 | 11.73 | 15.71 | 0 | 9.46 | 14.89 |
| 2 | E. coli | 47.34 | 30.30 | 50.26 | 42.10 | 25.02 | 44.46 | 35.49 | 22.16 | 41.06 | 33.34 | 14.35 | 34.30 |
| 3 | P. aurignosa | 41.84 | 32.08 | 46.17 | 35.99 | 21.16 | 40.28 | 24.47 | 16.74 | 31.13 | 14.42 | 10.90 | 21.93 |
| | | Concentration 2.18 g | | | | | | | | | | | |
| | | C | T | I | C | T | I | C | T | I | C | T | I |
| | | 20.0 | 1.8 | 21.8 | 2.0 | 0.18 | 2.18 | 0.2 | 0.018 | 0.218 | 0.02 | 0.0018 | 0.0218 |
| 1 | Klebsiella | 51.22 | 38.71 | 51.42 | 47.47 | 32.07 | 50.20 | 46.30 | 31.50 | 47.64 | — | — | — |
| 2 | C. albicanus | 37.55 | 35.47 | 40.46 | 31.25 | 26.45 | 34.47 | 18.37 | 15.65 | 25.22 | 12.90 | 0 | 14.09 |
| 3 | Stapylococcus | 50.93 | 36.96 | 54.27 | 35.76 | 27.49 | 37.38 | 22.45 | 16.68 | 25.57 | — | — | — |
| 4 | MRSA | 44.93 | 17.13 | 45.97 | 27.32 | 14.61 | 28.03 | — | — | — | — | — | — |

Where:
C = Ceftizidime;
T = Tobramycin;
I = Invention

Average hospitalization time of conventional treatment using ceftazidime before or after tobramycin was 14 to 21 days. In conventional treatment, Tobramycin was given at 40 mg to 80 mg bd and Ceftazidime 1 g to 2 g bd for 14-21 days.

Average hospitalization time for treatment of this invention is reduced to 25%. Due to reduced hospitalization and treatment time cost to patient/treatment is reduced.

It is obvious that with less treatment time and decreased hospitalization time, cost of treatment was less and relief to the patient was significantly improved with treatment of this invention.

### EXAMPLE 2

The composition of this invention was subjected to accelerated stability test. All procedures were carried out as per Standard Testing Procedures. The results show that the compositions of this invention of tobramycin and ceftazidime are stable.

TABLE 2
Accelerated stability data:
Name of the product: Ceftazidime & Tobramycin for Inj. 1.120 g
Strength: Each vial contains: Ceftazidime(Present as sterile Ceftazidime) 1.000 gm
Tobramycin (Present as Sterile Tobramycin) 0.120 gm
Batch NO: CFTB/T/08
Date of Mfg: July 2005
Date of Exp.: June 2007
Date of initiating: 03/07/2005
Packaging: 20 ml glass vial

| | | | | | | | | Assay (90.0-110.0% of | |
|---|---|---|---|---|---|---|---|---|---|
| Period | | | | | | BET NMT | | labelled) | |
| (Months) | Storage Condition | Description | Identification | Particulate matter | pH (5.0-8.0) | 0.10 EU/mg | Sterility | Ceftazidime | Tobramycin |
| Initial | — | A white | Complies | Complies | 6.65 | Complies | Complies | 100.3 | 99.8 |
| | | Crystalline | | | | | | | |
| | | powder | | | | | | | |
| 1 | 40° c., RH 75% | Almost | Complies | Complies | 6.60 | Complies | Complies | 99.7 | 98.0 |
| | | white | | | | | | | |
| | | Crystalline | | | | | | | |
| | | powder | | | | | | | |
| 2 | 40° c., RH 75% | Almost | Complies | Complies | 6.52 | Complies | Complies | 98.9 | 97.5 |
| | | white | | | | | | | |
| | | Crystalline | | | | | | | |
| | | powder | | | | | | | |
| 3 | 40° c., RH 75% | Light pale | Complies | Complies | 6.48 | Complies | Complies | 98.1 | 96.5 |
| | | yellow | | | | | | | |
| | | Crystalline | | | | | | | |
| | | powder | | | | | | | |
| 6 | 40° c., RH 75% | pale yellow | Complies | Complies | 6.41 | Complies | Complies | 97.3 | 96.1 |
| | | Crystalline | | | | | | | |
| | | powder | | | | | | | |

REMARKS:
1. All procedures carried out as per STP.
2. Above results shows that Product is stable at 40° c., RH 75% for 6 months.

### Accelerated Stability Data Report Sheet

Name of the product: Ceftazidime & Tobramycin for Inj. 2.180 g
Strength: Each vial contains: Ceftazidime(Present as sterile Ceftazidime) 2.000 gm
Tobramycin (Present as Sterile Tobramycin) 0.180 gm
Batch NO: CFTB/T/07
Date of Mfg: July 2005
Date of Exp.: June 2007
Date of initiating: 02/07/2005
Packaging: 30 ml glass vial

| | | | | | | | | Assay (90.0-110.0% | |
|---|---|---|---|---|---|---|---|---|---|
| Period | | | | | | BET NMT | | of labelled) | |
| (Months) | Storage Condition | Description | Identification | Particulate matter | pH (5.0-8.0) | 0.10 EU/mg | Sterility | Ceftazidime | Tobramycin |
| Initial | — | A white | Complies | Complies | 6.59 | Complies | Complies | 100.1 | 99.6 |
| | | Crystalline | | | | | | | |
| | | powder | | | | | | | |
| 1 | 40° c., RH 75% | Almost white | Complies | Complies | 6.47 | Complies | Complies | 99.6 | 98.8 |
| | | Crystalline | | | | | | | |
| | | powder | | | | | | | |
| 2 | 40° c., RH 75% | Almost white | Complies | Complies | 6.40 | Complies | Complies | 98.7 | 97.9 |
| | | Crystalline | | | | | | | |
| | | powder | | | | | | | |
| 3 | 40° c., RH 75% | Light pale | Complies | Complies | 6.29 | Complies | Complies | 98.0 | 97.1 |
| | | yellow | | | | | | | |
| | | Crystalline | | | | | | | |
| | | powder | | | | | | | |
| 6 | 40° c., RH 75% | pale yellow | Complies | Complies | 6.08 | Complies | Complies | 97.1 | 97.0 |
| | | Crystalline | | | | | | | |
| | | powder | | | | | | | |

REMARKS:
1. All procedures carried out as per STP.
2. Above results shows that Product is stable at 40° c. for 6 months.

### Accelerated Stability Data Report Sheet

Name of the product: Ceftazidime & Tobramycin for Inj. 560 mg
Strength: Each vial contains: Ceftazidime(Present as sterile Ceftazidime) 500.00 mg
Tobramycin (Present as Sterile Tobramycin) 060.00 mg
Batch NO: CFTB/T/09
Date of Mfg: July 2005
Date of Exp.: June 2007
Date of initiating: 04/07/2005
Packaging: 10 ml glass vial

| | | | | | | | | Assay (90.0-110.0% | |
|---|---|---|---|---|---|---|---|---|---|
| Period | | | | | | BET NMT | | of labelled) | |
| (Months) | Storage Condition | Description | Identification | Particulate matter | pH (5.0-8.0) | 0.10 EU/mg | Sterility | Ceftazidime | Tobramycin |
| Initial | — | A white | Complies | Complies | 6.62 | Complies | Complies | 100.0 | 99.9 |
| | | Crystalline | | | | | | | |
| | | powder | | | | | | | |
| 1 | 40° c., RH 75% | Almost white | Complies | Complies | 6.59 | Complies | Complies | 99.6 | 98.6 |
| | | Crystalline | | | | | | | |
| | | powder | | | | | | | |
| 2 | 40° c., RH 75% | Almost white | Complies | Complies | 6.53 | Complies | Complies | 98.5 | 97.9 |
| | | Crystalline | | | | | | | |
| | | powder | | | | | | | |
| 3 | 40° c., RH 75% | Light pale | Complies | Complies | 6.45 | Complies | Complies | 98.0 | 97.1 |
| | | yellow | | | | | | | |
| | | Crystalline | | | | | | | |
| | | powder | | | | | | | |
| 6 | 40° c., RH 75% | pale yellow | Complies | Complies | 6.39 | Complies | Complies | 97.0 | 96.6 |
| | | Crystalline | | | | | | | |
| | | powder | | | | | | | |

REMARKS:
1. All procedures carried out as per STP.
2. Above results shows that Product is stable at 40° c., RH 75% for 6 months.

### EXAMPLE 3

### Method of Making Liquid Composition

EDTA was dissolved in Water for injection. Sodium meta bi sulphite was added to this solution with continuous stirring and nitrogen purging (solution-1).

A buffer of 0.017M Sodium Citrate and 0.01 M Citric acid of pH 5.8 was added to solution-1 to make solution-2

Tobramycin and Ceftazidime were added one by one to solution-2 with continuous stirring below 25 degree celcius.

Phenol was added with continuous stirring and nitrogen purging. The volume was made up with water for injection and pH was readjusted whenever required.

Charcoal treatment during filtration with 0.2 micron is required to get colourless solution

1. A single formulation of the antibiotics, comprising:
tobramycin and ceftazidime in a synergistic combination;
wherein the single formulation is a chemically stabilized, safe and effective pharmaceutical composition.

2. The formulation as claimed in claim 1, wherein the formulation comprises a dry powder premix of the antibiotics and at least one chemical stabilizing agent for reconstitution with a parenterally acceptable diluent for in vivo use.

3. The formulation as claimed in claim 1, further comprising a liquid for injection for in vivo use.

4. The formulation as claimed in claim 1, further comprising a chemical stabilizing agent, wherein tobramycin and ceftazidime, pharmaceutically acceptable salts thereof, or a combination thereof are present in a weight ratio of between 1:8 to 1:11.

5. The formulation as claimed in claim 4, wherein the chemical stabilizing agent comprises an agent selected from the group consisting of sodium carbonate, L-arginine, L-methionine, L-histidine, L-cysteine and combinations thereof the like.

6. The formulation as claimed in claim 2, wherein the parenterally acceptable diluent comprises water containing a chelating agent selected from the group consisting of EDTA, salts thereof, and combinations thereof.

7. The formulation of claim 2 wherein:
a. the said dry powder premix for injection comprises of a two drug combination of tobramycin or a pharmaceutically acceptable salt thereof, in an amount of 20 to 220 mg as free acid form, and ceftazidime or a pharmaceutically acceptable salt thereof, in an amount of 250 mg to 2 gram as free acid form, the two drug combination taken in a weight/weight proportion of tobramycin:ceftazidime in a range of 1:8 to 1:11; and
b. the chemical stabilizing agent comprises an agent selected from the group consisting of L-arginine, sodium carbonate, L-cysteine, L-methionine, L-histidine, and combinations thereof.

8. The formulation of claim 3, wherein the said liquid for injection is added in a range of about 2 ml to 20 ml and comprises water, propylene glycol, polyethylene glycol, ethyl alcohol, or a combination thereof.

9. A method of treating infections in a host comprising administering to said host an antibiotically effective amount of the formulation according to claim 1.

10. A pharmaceutical formulation comprising:
about 60 mg to about 180 mg tobramycin; and
about 500 mg to about 2000 mg ceftizidime,
wherein a combination of tobramycin and ceftizidime exhibits an improved efficacy against at least one microorganism selected from the group consisting of MRSA, E. coli, P aurignosa, Klebisella, C. albicans, Staphylococcus and combinations thereof, as compared to a comparable amount of tobramycin alone and a comparable amount of ceftizidine alone.

11. The formulation of claim 7 wherein the two drug combination is taken in a weight/weight proportion of tobramycin:ceftazidime in a range of 1:8.33 to 1:11.2.

12. The formulation of claim 7 wherein the chemical stabilizing agent comprises an agent selected from the group consisting of L-arginine, sodium carbonate and combinations thereof.

13. The formulation of claim 12, wherein formulation comprises the antibiotics and the chemical stabilizing agent in a weight/weight proportion of tobramycin to ceftazidime to stabilizing agent in a ratio of 1:7:1, 1:8:1 to 1:10:1 or 1:9.8:1.3.

14. The pharmaceutical formulation of claim 10, further comprising a liquid for injection for in vivo use.

15. The pharmaceutical formulation of claim 10, comprising:
about 0.0006 mg/ml to about 1.8 mg/ml tobramycin; and
about 0.005 mg/ml to about 20 mg/ml ceftizidime.
